(19) United States
(12) Patent Application Publication
(10) Pub. No.: US 2004/0029191 A1
(43) Pub. Date: Feb. 12, 2004

(54) Detection of kinase substrates and products using ion mass spectrometry

(75) Inventors: Lee Lomas, Foster City, CA (US); Vanitha Thulasiraman, San Jose, CA (US); Tai-Tung Yip, Cupertino, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Ciphergen Biosystems, Inc., Fremont, CA

(21) Appl. No.: 10/440,042

(22) Filed: May 16, 2003

Related U.S. Application Data
(60) Provisional application No. 60/395,479, filed on Jul. 12, 2002.
(60) Provisional application No. 60/381,538, filed on May 17, 2002.

(51) Int. Cl. G01N 33/53; G01N 33/542

(52) U.S. Cl. 435/7.9

(57) ABSTRACT

The present invention provides materials and methods for simultaneously analyzing multiple kinase substrates and products using mass spectrometry.

### CROSS-REFERENCES TO RELATED APPLICATIONS

This application is related to U.S. provisional patent application No. 60/381,538, filed May 17, 2002, and provisional patent application No. 60/395,479, filed Jul. 12, 2002.

### REFERENCE TO A “SEQUENCE LISTING,” OR A COMPUTER PROGRAM LISTING APPENDIX SUBMITTED ON A COMPACT DISK

Not applicable.

### BACKGROUND OF THE INVENTION

This invention is directed to a method of simultaneously detecting the activity of several different kinases in a sample using mass spectroscopy [MS].

Kinases are important enzymes and they play important roles in signal transduction. Signal transduction refers to a process by which an external signal is transmitted into a cell to stimulate or inhibit intracellular processes.

Some components in the signal transduction pathways play roles in disease processes, such as cancer, allergy, arthritis, osteoporosis, and Alzheimer's disease. For example, in cancer cells, mutated versions of oncogenes and tumor suppressor genes, which are often components of signaling pathways that regulate cell growth and survival, result in uncontrollable growth of cancer cells. In another example, Alzheimer's disease involves altered regulation of various signal transduction pathway components, such as G-protein stimulated adenylyl cyclase, Ins(1,4,5)P₃ receptor, and protein kinase C.

Current technologies, such as Western blot or two-dimensional gel analyses, do not provide the speed, sensitivity or ability to analyze multiple kinase substrates at the same time. Therefore, there is a need to develop simpler analytical methods and materials for analyzing kinase substrate components for determining which kinases are contributing to a cellular function or disease process. Embodiments of the invention meet this and other goals.

### SUMMARY OF THE INVENTION

This invention provides for a method for detecting, measuring or monitoring the activity of a plurality of different kinases comprising first obtaining a plurality of affinity molecules, each affinity molecule specific for a particular protein kinase. A sample containing kinases is then contacted with the affinity molecules, so that the protein kinases of the sample are bound to the affinity molecules. The bound protein kinases are then immobilized on a solid support through the associated affinity molecule. To separate unbound material from the protein kinases, immobilized protein kinases are then washed. Each of the washed, immobilized protein kinases are then contacted with a substrate recognized by the respective kinase, and the kinase is allowed to convert the substrate to a phosphorylated product with a unique mass signature. These phosphorylated products are then captured on a probe comprising an adsorbent surface that binds the phosphorylated products and detected by mass spectrometry. Detection of the mass signature of a given phosphorylated product indicates the presence of the respective protein kinase in the sample.

Aspects of this invention include having the affinity molecules immobilized on the solid supports before contacting the sample with the affinity molecules and waiting to contact the affinity molecules to the solid support until after the affinity molecules have been contacted with the sample. Another aspect involves having each solid support bear a plurality of different affinity molecules that each recognizes a different protein kinase. Other aspects have each solid support bear affinity molecules that recognize only one protein kinase.

Some approaches to the invention have the washed, immobilized protein kinases contacting a substrate recognized by the kinase with all of the immobilized protein kinases in a common flow path. In these approaches, the solid supports bearing affinity molecules recognizing a particular protein kinase are segregated in the flow path from the solid supports recognizing different protein kinases. Still other approaches contact the solid supports bearing affinity molecules recognizing a particular protein kinase with a substrate for the protein kinase with the solid supports in different flow paths. These different flow paths can take the form of having the solid supports segregated in different wells of a multi-well flow plate.

In some embodiments of the invention, affinity molecules are antibodies. Some embodiments using antibodies as affinity molecules use protein A or Protein G as the solid support to which the antibody is bound. Other embodiments utilize a solid support comprising a microparticle, or a plastic surface such as the wall of a plastic micro well, dish sheet.

Certain aspects of the invention use probes that have adsorbent surfaces comprising a metal atom chelate to capture the phosphorylated products created by the kinases. The metal atom chelate can comprise copper, nickel, zinc, or gallium as well as other metal atoms. In other aspects the probe comprises a plurality of addressable locations, with each addressable location comprising an adsorbent surface.

In those aspects of the invention where the affinity molecules are specific for a plurality of different protein kinases, the protein kinases can be components of a signal transduction pathway, including apoptosis pathways

The present invention also includes kits for detecting protein kinases in a sample. These kits typically comprise a number of solid supports, where each solid support has a means to bind, or having bound thereto, an affinity molecule specifically recognizing a different protein kinase; a plurality of substrates, with each substrate being recognized by a different protein kinase that is in turn recognized by one of the affinity molecules. These substrates are phosphorylated by one of the protein kinases, producing a phosphorylated product with a unique mass signature; and finally, the kits comprise a probe having an adsorbent surface that binds the phosphorylated products produced by the kinases.

Some of these kits also comprise instruction material that contains details for using the kit. Some kits have affinity molecules that are antibodies. In kits comprising a solid support for immobilizing the protein kinases, the solid supports have a means through which to bind the affinity molecules. Where the affinity molecules are antibodies, the solid support and means for binding them can comprise protein A or protein G.

Some of the kits comprise solid supports that have affinity molecules already bound to the support. Such kits may also comprise a multi-well flow plate with each well being capable of accepting the solid supports.

The kits can also have solid supports with each solid support bearing a plurality of different affinity molecules that each recognize a different protein kinase. Other embodiments of the kit have solid supports that recognize one protein kinase. Kits can comprise solid supports that are in stackable cartridges, and/or comprise a flow column capable of accepting the solid supports.

Aspects of the kits include probes with one or more adsorbent surface comprising a metal atom chelate. The metal atom chelate can comprise copper, nickel, zinc, or gallium, and other metals.

### BRIEF DESCRIPTION OF THE DRAWINGS

FIG. 1 illustrates a preferred method for capture of phosphorylated kinase substrates using an probe of the present invention, an IMAC-Ga ProteinChip® Arrays.

FIG. 2A is a schematic diagram of a preferred embodiment of a high-throughput multiplexed kinase assay. Briefly, Antibodies specific for individual kinases are captured onto reactive beads, either directly via a covalent chemistry or indirectly via protein A/G. Crude extract containing the kinase(s) of interest is then is passed over the antibody-coated beads and specifically captured. After washing to remove non-specific material, a substrate specific to the captured kinase is added in a buffer containing ATP/Mg and the phosphorylation reaction is allowed to proceed. Reaction mixture containing substrate and phosphorylated product is then transferred to an IMAC-Gallium ProteinChip array for phospho-product enrichment and detection by mass spectrometry.

FIG. 2B schematically depicts the various alternatives to constructing a protein kinase affinity matrix of the invention. The solid supports can be constructed with affinity molecules specific for a particular kinase, or alternatively, each solid support can be conjugated to a number of affinity molecules, each affinity molecule specific for a different kinase. When the solid supports can be constructed with affinity molecules specific for a particular kinase, they can either be mixed in a common chamber, or segregated into different chambers, the latter being useful when one or more kinases is capable of cross-phosphorylating the substrate of another kinase activity being monitored. This segregated approach can be done with the solid supports being in a common flow path, or separated so that each has a separate flow path, such as when the solid supports are segregated into the wells of a multi-well flow plate.

FIG. 2C shows a compartment comprising four different derivatized solid supports contacted with a sample comprising kinases. The kinases bind to a solid support derivatized with the specific antibody. Then, mass-differentiated, kinase-specific substrates are applied to the supports and allowed to react with the kinases, producing phosphorylated products if the kinases are captured and are active. Then the phosphorylated products are eluted from the compartment and applied to the surface of an affinity biochip, such as an IMAC-gallium biochip from Ciphergen Biosystems. The phosphorylated products preferentially bind to the metal chelate biochip.

FIG. 3A illustrates a typical mass spectrographs produced in a protein kinase Cα activity titration using the present invention. The peak labeled “S” is the mass of the unlabelled substrate. The peak labeled “P” is the substrate after phosporylation with protein kinase Cα. Seven mass determinations were made in this experiment. Each determination represents the product of a kinase reaction carried out under identical conditions, but with a different amount of protein kinase Cα in the reaction mix. The uppermost determination represents an assay carried out with the least amount of protein kinase Cα (0.004 ng/μl), with the lowermost determination representing an assay were the maximum amount of protein kinase Cα was used (4 ng/μl).

FIG. 3B illustrates graphically the average normalized intensity of the phosphorylated substrate mass peak for each of the mass determinations shown in FIG. 3 A).

FIGS. 3C and D illustrate a protein kinase C inhibitor peptide (Upstate #12-151) inhibition curve. FIG. 3C indicates the change in mass distribution that occurs as the concentration of protein kinase C inhibitor peptide in the reaction is increased. The average normalized intensity of the phosphorylated substrate peak for each protein kinase C inhibitor peptide concentration is depicted graphically in FIG. 3D. The assays were carried out using Biomek in 192-well bioprocessors.

FIG. 4 illustrates the protein kinase Cα activity in varying amounts of mouse brain lysate immunoprecipitates. Protein kinase Cα in wild type mouse (B6) brain lysates was immunoprecipitated (IP) with anti-protein kinase Cα antibody (M4, Upstate) and immobilized on protein G-coated beads. The kinase activity was monitored by incubating beads with kinase substrate and the reaction mixture was transferred to BioChip® IMAC-Ga arrays for enrichment. The assays were carried out using Biomek in 96-well silent screen plates, which allow solutions to pass through, but not the beads.

FIG. 5 is a comparison of SELDI-based kinase assays with conventional radioactivity assays. Briefly, brain lysates were prepared from mice that had either normal amounts of Fyn-Kinase (WT), no Fyn-kinase (KO) or Fyn-kinase (Mutant). The Fyn-kinase was immunoprecipitated from each lysate with anti-Fyn antibody/protein-G complex, and then subjected to the kinase assay using the c-Src substrate. Determination of Fyn kinase activity determined for each lysate using the conventional ³²P kinase assay.

FIGS. 6A-C illustrates the monitoring the progression of kinase assays over time. Mass determinations of phosphorylated products from individual kinase assays are depicted in FIG. 6(A). In FIG. 6B, the kinases and substrates were mixed in a common assay and the intensity of the mass peaks produced by the phosphorylated substrate of each kinase was determined from a common sample of the reaction mix. The lower panel of FIG. 6B is a control showing the lack of a phosphorylation product for GSK3β in the common reaction is due to inhibition of GSKβ by Atk1. FIG. 6C is a cartoon illustrating receptor-mediated protein kinase cascades involved in controlling apoptosis.

FIG. 7 illustrates the utility of the SELDI technique in monitoring the phosphorylation state of multiple phosphorylation sites in a single protein or peptide.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

“Gas phase ion spectrometer” refers to an apparatus that detects gas phase ions. Gas phase ion spectrometers include an ion source that supplies gas phase ions. Gas phase ion spectrometers include, for example, mass spectrometers, ion mobility spectrometers, and total ion current measuring devices. “Gas phase ion spectrometry” refers to the use of a gas phase ion spectrometer to detect gas phase ions.

“Mass spectrometer” refers to a gas phase ion spectrometer that measures a parameter that can be translated into mass-to-charge ratios of gas phase ions. Mass spectrometers generally include an ion source and a mass analyzer. Examples of mass spectrometers are time-of-flight, magnetic sector, quadrupole filter, ion trap, ion cyclotron resonance, electrostatic sector analyzer and hybrids of these. “Mass spectrometry” refers to the use of a mass spectrometer to detect gas phase ions.

“Laser desorption mass spectrometer” refers to a mass spectrometer that uses laser energy as a means to desorb, volatilize, and ionize an analyte.

“Tandem mass spectrometer” refers to any mass spectrometer that is capable of performing two successive stages of m/z-based discrimination or measurement of ions, including ions in an ion mixture. The phrase includes mass spectrometers having two mass analyzers that are capable of performing two successive stages of m/z-based discrimination or measurement of ions tandem-in-space. The phrase further includes mass spectrometers having a single mass analyzer that is capable of performing two successive stages of m/z-based discrimination or measurement of ions tandem-in-time. The phrase thus explicitly includes Qq-TOF mass spectrometers, ion trap mass spectrometers, ion trap-TOF mass spectrometers, TOF-TOF mass spectrometers, Fourier transform ion cyclotron resonance mass spectrometers, electrostatic sector—magnetic sector mass spectrometers, and combinations thereof.

“Mass analyzer” refers to a sub-assembly of a mass spectrometer that comprises means for measuring a parameter that can be translated into mass-to-charge ratios of gas phase ions. In a time-of-flight mass spectrometer the mass analyzer comprises an ion optic assembly, a flight tube and an ion detector.

“Ion source” refers to a sub-assembly of a gas phase ion spectrometer that provides gas phase ions. In one embodiment, the ion source provides ions through a desorption/ionization process. Such embodiments generally comprise a probe interface that positionally engages a probe in an interrogatable relationship to a source of ionizing energy (e.g., a laser desorption/ionization source) and in concurrent communication at atmospheric or subatmospheric pressure with a detector of a gas phase ion spectrometer.

Forms of ionizing energy for desorbing/ionizing an analyte from a solid phase include, for example: (1) laser energy; (2) fast atoms (used in fast atom bombardment); (3) high energy particles generated via beta decay of radionucleides (used in plasma desorption); and (4) primary ions generating secondary ions (used in secondary ion mass spectrometry). The preferred form of ionizing energy for solid phase analytes is a laser (used in laser desorption/ionization), in particular, nitrogen lasers, Nd-Yag lasers and other pulsed laser sources. “Fluence” refers to the energy delivered per unit area of interrogated image. A high fluence source, such as a laser, will deliver about 1 mJ/mm2 to 50 mJ/mm2. Typically, a sample is placed on the surface of a probe, the probe is engaged with the probe interface and the probe surface is struck with the ionizing energy. The energy desorbs analyte molecules from the surface into the gas phase and ionizes them.

Other forms of ionizing energy for analytes include, for example: (1) electrons that ionize gas phase neutrals; (2) strong electric field to induce ionization from gas phase, solid phase, or liquid phase neutrals; and (3) a source that applies a combination of ionization particles or electric fields with neutral chemicals to induce chemical ionization of solid phase, gas phase, and liquid phase neutrals.

“Probe” in the context of this invention refers to a device adapted to engage a probe interface of a gas phase ion spectrometer (e.g., a mass spectrometer) and to present an analyte to ionizing energy for ionization and introduction into a gas phase ion spectrometer, such as a mass spectrometer. A “probe” will generally comprise a solid substrate (either flexible or rigid) comprising a sample presenting surface on which an analyte is presented to the source of ionizing energy.

“Surface-enhanced laser desorption/ionization” or “SELDI” refers to a method of desorption/Ionization gas phase ion spectrometry (e.g., mass spectrometry) in which the analyte is captured on the surface of a SELDI probe that engages the probe interface of the gas phase ion spectrometer. In “SELDI MS,” the gas phase ion spectrometer is a mass spectrometer. SELDI technology is described in, e.g., U.S. Pat. No. 5,719,060 (Hutchens and Yip) and U.S. Pat. No. 6,225,047 (Hutchens and Yip).

“Surface-Enhanced Affinity Capture” or “S EAC” is a version of SELDI that involves the use of probes comprising an absorbent surface (a “SEAC probe”). “Adsorbent surface” refers to a surface to which is bound an adsorbent (also called a “capture reagent,” an “affinity molecule” or an “affinity reagent”). An adsorbent is any material capable of binding an analyte (e.g., a target polypeptide or nucleic acid). “Chromatographic adsorbent” refers to a material typically used in chromatography. Chromatographic adsorbents include, for example, ion exchange materials, metal chelators (e.g., nitriloacetic acid or iminodiacetic acid), immobilized metal chelates, hydrophobic interaction adsorbents, hydrophilic interaction adsorbents, dyes, simple biomolecules (e.g., nucleotides, amino acids, simple sugars and fatty acids) and mixed mode adsorbents (e.g., hydrophobic attraction/electrostatic repulsion adsorbents). “Biospecific adsorbent” refers an adsorbent comprising a biomolecule, e.g., a nucleic acid molecule (e.g., an aptamer), a polypeptide, a polysaccharide, a lipid, a steroid or a conjugate of these (e.g., a glycoprotein, a lipoprotein, a glycolipid, a nucleic acid (e.g., DNA)-protein conjugate). In certain instances the biospecific adsorbent can be a macromolecular structure such as a multiprotein complex, a biological membrane or a virus. Examples of biospecific adsorbents are antibodies, receptor proteins and nucleic acids. Biospecific adsorbents typically have higher specificity for a target analyte than chromatographic adsorbents. Further examples of adsorbents for use in SELDI can be found in U.S. Pat. No. 6,225,047 (Hutchens and Yip, “Use of retentate chromatography to generate difference maps,” May 1, 2001).

In some embodiments, a SEAC probe is provided as a pre-activated surface which can be modified to provide an adsorbent of choice. For example, certain probes are provided with a reactive moiety that is capable of binding a biological molecule through a covalent bond. Epoxide and carbodiimidizole are useful reactive moieties to covalently bind biospecific adsorbents such as antibodies or cellular receptors.

“Adsorption” refers to detectable non-covalent binding of an analyte to an adsorbent or capture reagent.

“Surface-Enhanced Neat Desorption” or “SEND” is a version of SELDI that involves the use of probes comprising energy absorbing molecules chemically bound to the probe surface. (“SEND probe.”) “Energy absorbing molecules” (“EAM”) refer to molecules that are capable of absorbing energy from a laser desorption/ionization source and thereafter contributing to desorption and ionization of analyte molecules in contact therewith. The phrase includes molecules used in MALDI, frequently referred to as “matrix”, and explicitly includes cinnamic acid derivatives, sinapinic acid (“SPA”), cyano-hydroxy-cinnamic acid (“CHCA”) and dihydroxybenzoic acid, ferulic acid, hydroxyacetophenone derivatives, as well as others. It also includes EAMs used in SELDI. In certain embodiments, the energy absorbing molecule is incorporated into a linear or cross-linked polymer, e.g., a polymethacrylate. For example, the composition can be a co-polymer of α-cyano-4-methacryloyloxycinnamic acid and acrylate. In another embodiment, the composition is a co-polymer of α-cyano-4-methacryloyloxycinnamic acid, acrylate and 3-(tri-methoxy)silyl propyl methacrylate. In another embodiment, the composition is a co-polymer of α-cyano-4-methacryloyloxycinnamic acid and octadecylmethacrylate (“C18 SEND”). SEND is further described in U.S. Pat. No. 5,719,060 and U.S. patent application No. 60/408,255, filed Sep. 4, 2002 (Kitagawa, “Monomers And Polymers Having Energy Absorbing Moieties Of Use In Desorption/Ionization Of Analytes”).

“Surface-Enhanced Photolabile Attachment and Release” or “SEPAR” is a version of SELDI that involves the use of probes having moieties attached to the surface that can covalently bind an analyte, and then release the analyte through breaking a photolabile bond in the moiety after exposure to light, e.g., laser light. SEPAR is further described in U.S. Pat. No. 5,719,060.

“Eluant” or “wash solution” refers to an agent, typically a solution, which is used to affect or modify adsorption of an analyte to an adsorbent surface and/or remove unbound materials from the surface. The elution characteristics of an eluant can depend, for example, on pH, ionic strength, hydrophobicity, degree of chaotropism, detergent strength and temperature.

“Analyte” refers to any component of a sample that is desired to be detected. The term can refer to a single component or a plurality of components in the sample.

The “complexity” of a sample adsorbed to an adsorption surface of an affinity capture probe means the number of different protein species that are adsorbed.

“Molecular binding partners” and “specific binding partners” refer to pairs of molecules, typically pairs of biomolecules that exhibit specific binding. Molecular binding partners include, without limitation, receptor and ligand, antibody and antigen, biotin and avidin, and biotin and streptavidin.

“Monitoring” refers to recording changes in a continuously varying parameter.

“Solid support” refers to a solid material which can be derivatized with, or otherwise attached to, a chemical moiety, such as a capture reagent, a reactive moiety or an energy absorbing species. Exemplary solid supports include chips (e.g., probes), microtiter plates and chromatographic resins.

“Chip” refers to a solid support having a generally planar surface to which a chemical moiety may be attached. Chips that are adapted to engage a probe interface are also called “probes.”

“Biochip” refers to a chip to which a chemical moiety is attached. Frequently, the surface of the biochip comprises a plurality of addressable locations, each of which location has the chemical moiety attached there.

“Protein biochip” refers to a biochip adapted for the capture of polypeptides. Many protein biochips are described in the art. These include, for example, protein biochips produced by Ciphergen Biosystems (Fremont, Calif.), Packard BioScience Company (Meriden Conn.), Zyomyx (Hayward, Calif.) and Phylos (Lexington, Mass.). Examples of such protein biochips are described in the following patents or patent applications: U.S. Pat. No. 6,225,047 (Hutchens and Yip, “Use of retentate chromatography to generate difference maps,” May 1, 2001); International publication WO 99/51773 (Kuimelis and Wagner, “Addressable protein arrays,” Oct. 14, 1999); U.S. Pat. No. 6,329,209 (Wagner et al., “Arrays of protein-capture agents and methods of use thereof,” Dec. 11, 2001) and International publication WO 00/56934 (Englert et al., “Continuous porous matrix arrays,” Sep. 28, 2000).

Protein biochips produced by Ciphergen Biosystems comprise surfaces having chromatographic or biospecific adsorbents attached thereto at addressable locations. Ciphergen ProteinChip® arrays include NP20, H4, H50, SAX-2, Q-10, WCX-2, CM-10, IMAC-3, IMAC-30, LSAX-30, LWCX-30, IMAC-40, PS-10, PS-20 and PG-20. These protein biochips comprise an aluminum substrate in the form of a strip. The surface of the strip is coated with silicon dioxide.

In the case of the NP-20 biochip, silicon oxide functions as a hydrophilic adsorbent to capture hydrophilic proteins.

H4, H50, SAX-2, Q-10, WCX-2, CM-10, IMAC-3, IMAC-30, PS-10 and PS-20 biochips further comprise a functionalized, cross-linked polymer in the form of a hydrogel physically attached to the surface of the biochip or covalently attached through a silane to the surface of the biochip. The H4 biochip has isopropyl functionalities for hydrophobic binding. The H50 biochip has nonylphenoxy-poly(ethylene glycol)methacrylate for hydrophobic binding. The SAX-2 and Q-10 biochips have quaternary ammonium functionalities for anion exchange. The WCX-2 and CM-10 biochips have carboxylate functionalities for cation exchange. The IMAC-3 and IMAC-30 biochips have nitriloacetic acid functionalities that adsorb transition metal ions, such as Cu++ and Ni++, by chelation. These immobilized metal ions allow adsorption of peptide and proteins by coordinate bonding. The PS-10 biochip has carboimidizole functional groups that can react with groups on proteins for covalent binding. The PS-20 biochip has epoxide functional groups for covalent binding with proteins. The PS-series biochips are useful for binding biospecific adsorbents, such as antibodies, receptors, lectins, heparin, Protein A, biotin/streptavidin and the like, to chip surfaces where they function to specifically capture analytes from a sample. The PG-20 biochip is a PS-20 chip to which Protein G is attached. The LSAX-30 (anion exchange), LWCX-30 (cation exchange) and IMAC-40 (metal chelate) biochips have functionalized latex beads on their surfaces. Such biochips are further described in: WO 00/66265 (Rich et al., “Probes for a Gas Phase Ion Spectrometer,” Nov. 9, 2000); WO 00/67293 (Beecher et al., “Sample Holder with Hydrophobic Coating for Gas Phase Mass Spectrometer,” Nov. 9, 2000); U.S. patent application US 2003 0032043 A1 (Pohl and Papanu, “Latex Based Adsorbent Chip,” Jul. 16, 2002) and U.S. patent application No. 60/350,110 (Um et al., “Hydrophobic Surface Chip,” Nov. 8, 2001); U.S. patent application No. 60/367,837, (Boschetti et al., “Biochips With Surfaces Coated With Polysaccharide-Based Hydrogels,” May 5, 2002) and U.S. patent application entitled “Photocrosslinked Hydrogel Surface Coatings” (Huang et al., filed Feb. 21, 2003).

Upon capture on a biochip, analytes can be detected by a variety of detection methods selected from, for example, a gas phase ion spectrometry method, an optical method, an electrochemical method, atomic force microscopy and a radio frequency method. Gas phase ion spectrometry methods are described herein. Of particular interest is the use of mass spectrometry and, in particular, SELDI. Optical methods include, for example, detection of fluorescence, luminescence, chemiluminescence, absorbance, reflectance, transmittance, birefringence or refractive index (e.g., surface plasmon resonance, ellipsometry, a resonant mirror method, a grating coupler waveguide method or interferometry). Optical methods include microscopy (both confocal and non-confocal), imaging methods and non-imaging methods. Immunoassays in various formats (e.g., ELISA) are popular methods for detection of analytes captured on a solid phase. Electrochemical methods include voltametry and amperometry methods. Radio frequency methods include multipolar resonance spectroscopy.

The term “addressable location” refers to a position that can be fixed in space within defined limits and accessible through an index unique for the position.

The term “apoptosis pathway” refers to an active process requiring metabolic activity by the dying cell, often characterized by systematic cleavage of cellular DNA and disintegration of cells into membrane-bound particles.

In the context of the present invention, the term “common flow path” refers to a single course of fluid flow shared by two or more solid supports.

In the context of the present invention, the phrase “immobilized protein kinases” refers to protein kinases bound by affinity molecules attached to a solid support.

The term “mass signature” refers to the pattern of molecular mass signals produced by a single molecule. The mass signature is generally dominated by a single molecular mass signal, although fragmentation caused by the physical characteristics of the analysis process can produce one or more less pronounced satellite peaks.

The term “metal atom chelate” refers to a substance or a surface comprising metal atoms capable of acting as nuclei for the formation of chelation complexes with suitable molecules. In the context of the present invention, the metal atom chelate preferentially forms chelation complexes with phosphorylated molecules, typically polypeptides and proteins.

The term “microparticle” refers to very small spheroid particles of a core composition that is insoluble in aqueous solution and usually capable of forming aqueous suspensions. Exemplary microparticle core compositions include various organic polymers (e.g., plastics and latex) polysaccharides, proteins, metals, carbon, and silica. Microparticles can range in size from 0.01 μm to 10 μm in diameter, preferably between 0.05 μm and 3 μm in diameter, more preferably between 0.1 μm and 1 μm in diameter. The term “microparticle” further includes coatings made on the core composition, whether bonded covalently or non-covalently. Exemplary coatings include proteinaceous deposits, metallic films, nucleic acids, carbohydrates and polysaccharides, and lipid layers.

The term “phosporylation” refers to the covalent modification of a molecule by the addition of a phosphoryl group, usually catalyzed by a an enzyme termed a “protein kinase.”

In the context of the present invention, the term “phosphorylated products” refers to the modified biomolecules, usually a polypeptide or protein, produced through the covalent addition of one or more phosphoryl groups to a protein kinase substrate by the respective protein kinase.

The term “plastic surface” refers to the interface between any organic polymer and the surrounding environment. Examples of articles having “plastic surfaces” include plastic sheets, bowls, and the surfaces of multi-well plates.

The term “protein A” and refers to a highly stable surface receptor produced by Staphylococcus aureus, which is capable of binding the Fc portion of immunoglobulins, especially IgGs, from a large number of species (Boyle, M. D. P. and K. J. Reis. Bacterial Fc Receptors. Biotechnology 5:697-703 (1987).). One protein A molecule can bind at least 2 molecules of IgG simultaneously (Sjöquist, J., Meloun, B. and Hjelm, H. Protein A isolated from Staphylococcus aureus after digestion with lysostaphin. Eur J Biochem 29: 572-578 (1972)).

The term “protein G” refers to a cell surface-associated protein from streptococcus that binds to IgG with high affinity. It has three highly homologous IgG-binding domains. (See Lian, et.al. 1992. Journal of Mol. Biol. 228:1219-1234 and Derrick and Wigley. 1994. Journal of Mol. Biol. 243:906-918.)

The term “protein kinase” Refers to any protein or protein derivative capable of catalyzing the covalent addition of a phosphoryl group to a protein, a process termed “phosphorylation.”

The term “sample” refers to any substance originally derived, or partially derived, from a cellular source and containing molecules with biological activity or function.

In the context of the present invention, the term “stackable cartridges” refers to discrete individual units capable of being combined with similar discrete individual units to form a functional composite. The functional composite formed can have a common flow path or be combined to form a device with multiple independent flow paths.

In the context of the present invention, the term “unbound material” refers to any material that is not bound by an affinity molecule and capable of being transported unbound by affinity molecules present through simple fluid flow.

The term “wells”, in the context of a multi-well plate, refers to the individual compartments of the plate capable of segregating their contents from the contents of other wells found on the same plate or other apparatus.

The term “antibody” refers to a polypeptide ligand substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad immunoglobulin variable region genes. Antibodies exist, e.g., as intact immunoglobulins or as a number of well-characterized fragments produced by digestion with various peptidases. This includes, e.g., Fab′ and F(ab)′₂ fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH₁, CH₂ and CH₃, but does not include the heavy chain variable region.

Methods for preparing antibodies are well-known in the art. See, e.g., Coligan, Current Protocols in Immunology (1991); Harlow & Lane, Antibodies: A Laboratory Manual (1988); Goding, Monoclonal Antibodies: Principles and Practice (2d ed. 1986); and Kohler & Milstein, Nature 256:495-497 (1975). Such techniques include, but are not limited to, antibody preparation by selection of antibodies from libraries of recombinant antibodies in phage or similar vectors, as well as preparation of polyclonal and monoclonal antibodies by immunizing rabbits or mice (see, e.g., Huse et al., Science 246:1275-1281 (1989); Ward et al., Nature 341:544-546 (1989)).

A “control amount” of a kinase substrate component can be any amount or a range of amount which is to be compared against a test amount of the component. For example, a control amount of the component can be the amount of the component in a normal cell or person, which or who is known to have an intact and functional level of the kinase being studied in either an absolute amount (e.g., pg/ml) or a relative amount (e.g., relative intensity of signals).

“Detect” refers to identifying the presence, absence or amount of the object to be detected.

The phrase “differentially present” refers to differences in the quantity of a kinase substrate component present in a test sample as compared to a control (a sample taken from a normal subject or cells). A kinase substrate component is differentially present between the two samples if the amount of the component in one sample is statistically significantly different from the amount of the polypeptide in the other sample. For example, a polypeptide is differentially present between the two samples if it is present at least about 120%, at least about 130%, at least about 150%, at least about 180%, at least about 200%, at least about 300%, at least about 500%, at least about 700%, at least about 900%, or at least about 1000% greater than it is present in the other sample, or if it is detectable in one sample and not detectable in the other.

The term “kinase substrate” or simply “substrate” refers to a molecule that can be phosphorylated by a kinase. Once phosphorylated, the molecule is termed product and both the product and the substrate may be detectable using MS. To the extent that this document refers to kinase substrates that have been exposed to kinase enzymes, it is understood that the term substrate would also include phosphorylated substrate or product without further explanation. The combination of substrate and product is also referred to as kinase substrate components.

“Signal transduction” refers to a process by which the information contained in an extracellular physical or chemical signal (e.g., hormone or growth factor) is received by the cell by the activation of specific receptors and conveyed across the plasma membrane, and along an intracellular chain of various components, to stimulate the appropriate cellular response.

“Signal transduction pathway components,” “pathway components,” or “components of a signal transduction pathway” refer to intracellular or transmembrane biomolecules (of a particular apparent molecular weight) which are activated in cascade in response to an extracellular signal received by the cell.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to Ras protein from specific species such as rat, mouse, or human can be selected to obtain only those polyclonal antibodies that are specifically immunoreactive with Ras protein and not with other proteins, except for polymorphic variants and alleles of Ras protein. This selection may be achieved by subtracting out antibodies that cross-react with Ras proteins from other species. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

“Sorbent” refers to any material that can specifically adsorb or absorb a specific kinase and hold it to a solid support.

A “test amount” of a signal transduction pathway component refers to an amount of the component present in a sample being tested. A test amount can be either in absolute amount (e.g., μg/ml) or in a relative amount (e.g., relative intensity of signals).

### DETAILED DESCRIPTION

I. Introduction

Cell signal transduction mediated by protein phosphorylation is the major mechanism through which cellular responses are regulated in response to environmental and metabolic changes. Cellular responses regulated by protein phosphorylation include; transcription of specific genes, cell growth, cell death, cell division, cell adhesion, endocytosis, etc. A particularly interesting application of the present invention is to the study of the role of phosporylation in apoptosis. A number of protein kinase activities have been implicated in apoptotic events initiated by a diverse set of stimuli. Such protein kinases include those of the JNK, MAP and CDK kinase families, as well as other serine/threonine and tyrosine kinase activities. Substrates suitable to the invention are readily available to those of skill in the art for all of these kinase activities, as are antibodies for both the kinases and substrates (e.g., for immobilization of kinases, affinity purification of substrates, or isolation of native phosphorylated substrates). For example, Biocat GmbH, Neuenheimer Feld 581, D-69120, Heidelberg, FRG, Tel: +49(0)6221-5858-44, cat. no. HM4000, can supply an apoptosis antibody array containing 150 antibodies specific for proteins, including protein kinases, implicated in apoptosis:

TABLE 1
Apoptosis Antibody Array ™
(Antibody List by Antigen Name)

| | |
|---|---|
| 1 | ACINUS |
| 2 | ACINUS-p23 |
| 3 | AFAP |
| 4 | AIF |
| 5 | Bin 1 |
| 6 | ANT |
| 7 | Apaf1 |
| 8 | APC |
| 9 | ARC |
| 10 | Bad |
| 11 | Bak |
| 12 | Bax |
| 13 | BOK |
| 14 | NBK |
| 15 | Bag-1 |
| 16 | Bcl-2 |
| 17 | Bcl-w |
| 18 | Bcl-xS/L |
| 19 | A1 |
| 20 | Mcl-1 |
| 21 | BID |
| 22 | Bim |
| 23 | BAP1 |
| 24 | BARD1 |
| 25 | BRCA1 |
| 26 | BRCA2 |
| 27 | BRUCE |
| 28 | CAS |
| 29 | Caspase1 |
| 30 | Caspase2 |
| 31 | Caspase3 |
| 32 | Caspase4 |
| 33 | Caspase5 |
| 34 | Caspase6 |
| 35 | Caspase7 |
| 36 | Caspase8 |
| 37 | Caspase9 |
| 38 | Caspase10 |
| 39 | CD27 |
| 40 | CD30 |
| 41 | CD40 |
| 42 | C-IAP1 |
| 43 | C-IAP2 |
| 44 | CIDE-A |
| 45 | CIDE-B |
| 46 | Clusterin |
| 47 | CPAN |
| 48 | cytochrome C |
| 49 | DAXX |
| 50 | DCC |
| 51 | DFF45/ICAD |
| 52 | DIVA |
| 53 | DMBT1 |
| 54 | DMC1 |
| 55 | DcR1 |
| 56 | DR2 |
| 57 | DR3 |
| 58 | DR4 |
| 59 | DR5 |
| 60 | Elongin A |
| 61 | FADD |
| 62 | FAF-1 |
| 63 | Fas |
| 64 | FasL |
| 65 | FAST |
| 66 | FHIT |
| 67 | FLASH |
| 68 | FLIPs/I |
| 69 | GADD34 |
| 70 | GADD45 |
| 71 | GADD153 |
| 72 | Granzyme B |
| 73 | hdlg |
| 74 | Ne-dlg |
| 75 | hlLP |
| 76 | Hrk |
| 77 | IEXs/l |
| 78 | ING1-p33 |
| 79 | Jak1 |
| 80 | Jak2 |
| 81 | Jak3 |
| 82 | JNK1,2,3 |
| 83 | p-JNK1,2,3 |
| 84 | LGI1 |
| 85 | Maspin |
| 86 | MDA-7 |
| 87 | MDM2 |
| 88 | MEF2 |
| 89 | MEK1 |
| 90 | MEKK1 |
| 91 | MEKK2 |
| 92 | Menin |
| 93 | MGMT |
| 94 | c-Myc |
| 95 | NF1GRP |
| 96 | NF2 |
| 97 | NF-kappa B 50 |
| 98 | NF-kappa B 52 |
| 99 | NF-kappa B p65 |
| 100 | lkappa B-a |
| 101 | lkappa B-b |
| 102 | lkappa B kinase a |
| 103 | lkappa B kinase b |
| 104 | Nibrin |
| 105 | NIK |
| 106 | nip1 |
| 107 | nip2 |
| 108 | nip3 |
| 109 | p53 |
| 110 | P63 |
| 111 | P73 |
| 112 | PAR-4 |
| 113 | PARP |
| 114 | PSD-95 |
| 115 | PTEN |
| 116 | Rad51 |
| 117 | Rad52 |
| 118 | RAIDD |
| 119 | Rb (p107) |
| 120 | Rb (p110) |
| 121 | RbP130 (Rb2) |
| 122 | RBBP |
| 123 | c-Rel |
| 124 | RICK |
| 125 | RIP |
| 126 | SIVA |
| 127 | SODD |
| 128 | Stat1 |
| 129 | SURVIVIN |
| 130 | TANK |
| 131 | TDAG51 |
| 132 | TIA-1 |
| 133 | TIAR |
| 134 | TNFR1 |
| 135 | TNFR2 |
| 136 | TOSO |
| 137 | TRADD |
| 138 | TRAF1 |
| 139 | TRAF2 |
| 140 | TRAF3 |
| 141 | TRAF4 |
| 142 | TRAF5 |
| 143 | TRAF6 |
| 144 | TRAIL |
| 145 | TSG101 |
| 146 | Tuberin |
| 147 | Tyk2 |
| 148 | VDAC1 |
| 149 | VHL |
| 150 | XRCC4 |

Protein phosphorylation reactions result in the addition of a phosphoryl group to a serine, threonine or tyrosine residue of a protein. Protein phosphorylation reactions are catalyzed by a group of enzymes called protein kinases. Protein kinases generally fall into two broad groups; the serine/threonine kinases and the tyrosine kinases, depending on which amino acid residue the kinase adds the phosphoryl group. The enzymes that remove the phosphoryl group from the protein, the protein phosphatases, are also broadly categorized based upon which amino acid residue the phosphoryl group is removed from.

Traditionally, protein phosphorylation has been studied by measuring changes in the incorporation of radioactive 32P/33P into specific protein substrates. Although highly sensitive, the use of a radioactivity assay in itself requires special materials management; the assay is of a single use; and it is impossible to multiplex kinase assays in a single reaction mixture without performing cumbersome separation techniques, which by their nature degrade the accuracy of the assay and create waste problems when performed with radioactive components.

Because of the prevalence of phosphorylation as a regulatory mechanism and the importance of the pathways controlled by this form of cellular signaling, monitoring kinase activities is a valuable means for measuring and studying the condition and state of cellular processes. Moreover, cell signaling by protein phosphorylation frequently involves numerous protein kinase activities, each interacting with another kinase or kinase regulator. These kinase networks, or phosporylation cascades, provide several extremely important functions to the signaling process. First, as one kinase in a cascade frequently acts as a substrate for another kinase in a cascade, kinase cascades offer signal amplification as one set of kinases becomes activated and phosphorylates another downstream set of kinases. Second, catalytic phosporylation by kinases can be an extremely rapid event, allowing for rapid propagation of the cellular signal. Third, activation of a single kinase in response to a cellular signal can effect a diverse set of processes, as the activated kinase can have multiple substrates, each ultimately impacting a different aspect of cellular metabolism. Important classes of protein kinases involved in cellular signal transduction include the serine/threonine kinases and the tyrosine kinases.

As protein phosphorylation plays such a central role in cellular signal transduction, determination of the activities of the enzymes involved in catalyzing phosphorylation events is critical to understanding cellular metabolism in both normal and disease states. An important feature of the present invention is the ability to monitor several kinase activities present at a particular time and under a particular set of conditions. This feature of the invention, termed multiplexing, allows for the determination of which kinases are involved in particular cellular responses and frequently allows for the determination of the temporal sequence of activation of the activities giving an indication of how the activities interact or otherwise behave in response to each other. In an exemplary study of protein kinases involved in the apoptosis pathway, the present invention was able to elucidate the relationship between the serine/threonine kinases Akt and GSK3, showing that Akt inactivates GSK3. By studying the relative activity of the two enzymes the activity characteristics of the overall metabolic pathway was determined. Other pathways that can be deciphered using the present invention include: cell cycling pathways, developmental pathways, and both anabolic and catabolic pathways.

It will also be noted by those of skill in the art that the present invention can easily be modified to detect the protein phosphatase activities that remove the phosphoryl group added to proteins by kinases. A presently large and rapidly growing body of literature documents the importance of phosphatases as points of regulation in signal transduction systems. Phosphatases are also critical in maintaining the turnover of protein-bound phosphate in proteins involved in cellular metabolism, including enzymes catalyzing the rate limiting steps of metabolic processes, as well as other phosphatases and kinases. The continuous turnover of protein-bound phosphate allows proteins whose activity is regulated in this manner to be highly sensitive to any change in the relative activities of the kinases and phosphatases catalyzing the phosphorylation/dephosphorylation reactions. This allows the cell to be highly sensitive to any signal altering the relative activities of kinases and phosphatases in response to a cellular or extracellular signal, and allowing for an extremely rapid cellular response.

Here we describe a method of monitoring protein kinase activity by surface enhanced laser desorption/ionization (SELDI) mass spectrometry. The method is highly sensitive, eliminates the necessity for the use of radioactivity, and can be easily multiplexed for the analysis of several different kinases in a single reaction mixture. We have compared the SELDI-based kinase assay to the conventional radioactivity assay by determining kinase activities in mouse brain lysates. In addition, we have used the SELDI kinase assay to determine EC₅₀ of kinase activities and IC₅₀ of kinase inhibitors in a high-throughput fashion. We have also used the technique in conjunction with MS/MS analysis to map phosphorylation sites.

Possible applications range from multiplexed, high throughput kinase assays for the identification of novel substrates or kinase inhibitors, to the analysis of the activity states of signal transduction pathways in disease states. Suitable kinases for such studies include enzymes from both the serine/threonine and tyrosine kinase classes, for example AKt1, JNK1α1, C-Src, GSK3β, protein kinases A and C, and members of the Aurora kinase family. Surface chemistries and capture reagents (antibodies) specifically recognizing these exemplary kinases are generally commercially available.

SELDI-MS also offers many advantages over other MS-based assay systems, such as MALDI. For example, sample preparation in MALDI analysis culminates in a single sample being placed on the probe. In contrast, SELDI analysis culminates in multiple samples being placed on a single probe, as each SELDI probe has multiple sample adsorbent areas each capable of comprising a different adsorbent chemistry. When inserted into a suitable mass spectrometer, each of these sample adsorbent areas behaves as a separate source of sample for analysis, allowing SELDI-MS to be used as a high throughput screening system. Moreover, unlike the probes used in MALDI analysis, the adsorbent chemistries of the SELDI probe retain the molecules of interest, allowing for concentration of the molecule of interest and removal of contaminants that can degrade the spectra. thus the ability to retain molecules of interest on a biochip increases the signal to noise ratio of the analysis. Comparable concentration and purification is not available in MALDI preparation without the inclusion of burdensome chromatography steps, and MALDI cannot afford the user the potential for high throughput analysis on a scale comparable to SELDI.

The following is an overview of the invention. In a first part of the invention, affinity media derivatized with antibodies against each kinase in a pathway are provided. They may be provided in a single compartment or in distinct compartments. These compartments could be separate wells of a multi-titer flow plate, or a single column partitioned into which the media are packed in different areas, possibly separated by frits. This second method provides certain advantages, as will be apparent.

A cell lysate or other appropriate sample is then passed over the affinity media, and the kinases in the sample captured by the respective derivatized antibodies specific for each protein kinase. If a multi-well plate is used, the sample does not have to be divided into aliquots as long as the substrates do not share a common mass. The single compartment or single partitioned column can also accommodate a single sample.

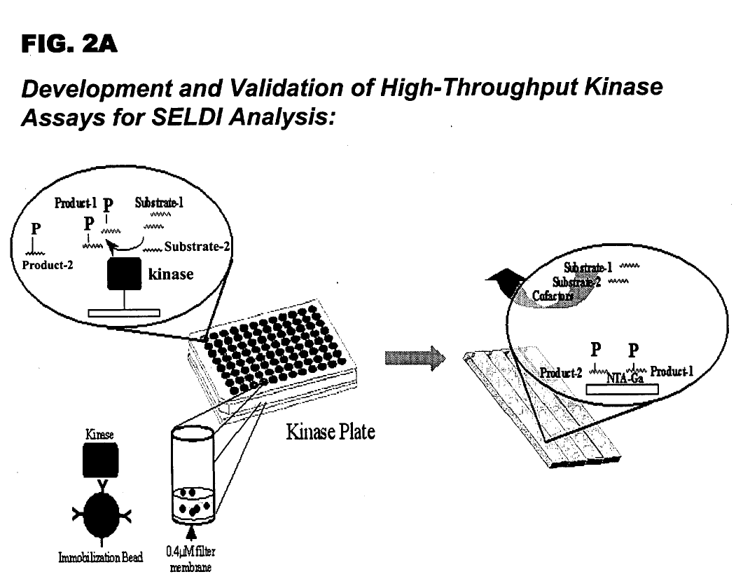

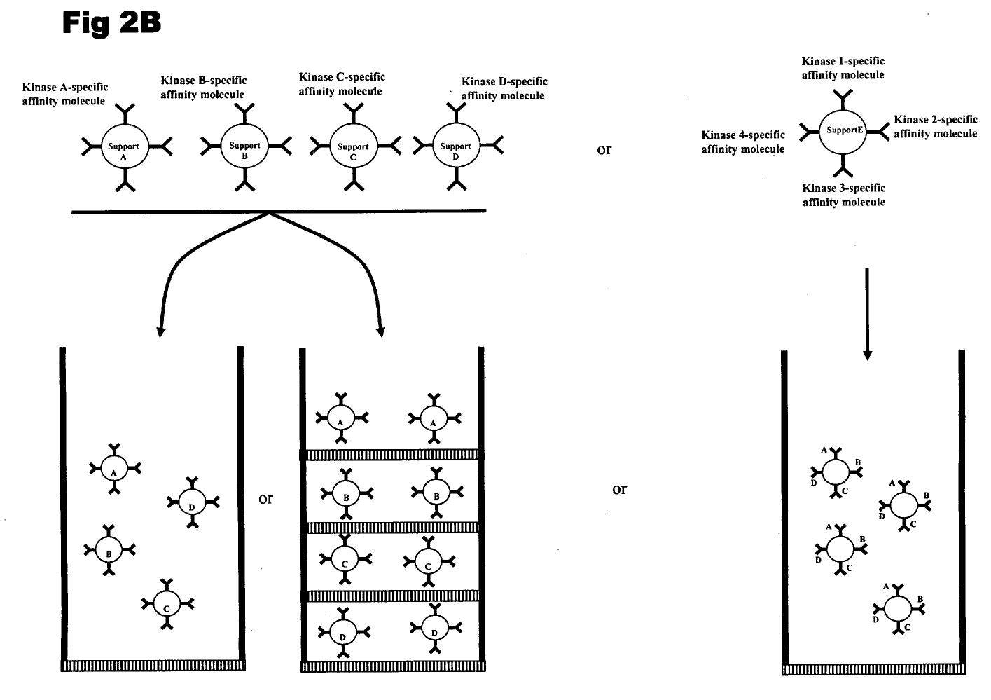

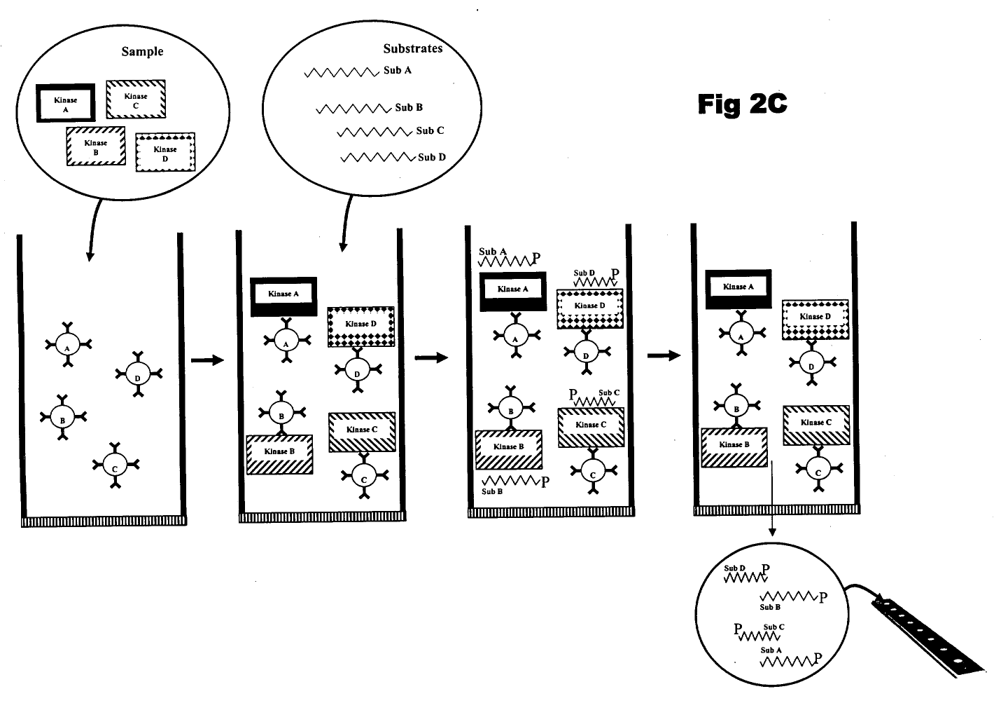

Substrates for each kinase that are mass-distinguishable and/or unique to each kinase are then passed over the column or the plate wells so as to allow the captured kinases to convert substrate into phosphorylated product. In the case of a column, the substrates must be unique to each kinase. As depicted in FIG. 2B, affinity columns of the invention can be constructed with a heterogenous mixture of beads with different affinities, beads that are heterospecific for different kinases, or constructed using layers of affinity beads, each segmented layer having beads that are specific for a particular kinase. In the case of individual wells, it is sufficient that substrates be mass-distinguishable because the substrates are exposed to only a single protein kinase activity. The products are then collected from the column or from the wells. Once phosphorylated, the products produced in wells can be pooled and analyzed as part of a common sample, or treated separately.

The products are applied to an probe having a substratum to which is attached one or more areas of adsorbent material that preferentially bind phosphorylated products. In a preferred embodiment, the adsorbent is a metal atom chelate, such as a gallium, as appears on the IMAC-Ga ProteinChip® Array (available from Ciphergen Biosystems, Inc., Fremont, Calif.), that captures phosphorylated products. The entire the kinase capture/substrate phosphorylation and isolation method is illustrated diagrammatically in FIG. 2C. Then, the captured products are detected by mass spectrometry. Because the products are mass distinguishable, one can distinguish them in a single sample by SELDI. Furthermore, because the mass of each product is associated with a particular kinase, the activity of that kinase can be gauged directly. This method is preferred to detecting the different products on a different probe because relative amounts of the phosphorylation products of each protein kinase can be compared directly. Alternatively, one could avoid pooling products collected from wells on the plate and test each one separately, however this introduces complications that the single probe method avoids. By determining which kinases in a sample are active, one can then determine which signal transduction pathways are active.

In one aspect of this invention, the parts of the invention are produced as “kits.” A kit can include, for example, a plurality of affinity media in which each medium is derivatized with an antibody that binds to a particular kinase in the pathway. The media could be packed in separate chambers, e.g., multiple wells of a 96-well flow plate. In another embodiment, the media could be packed in a partitioned column separated by, for example, a frit or filter paper. In certain embodiments, the partitioned segments are packaged in cassettes that can be assembled into a partitioned column of the invention by snapping or screwing the pieces together. If unique substrates are available, they can be applied to the partitioned column. If unique substrates are not available, then the column can be dis-assembled and substrates can be applied to each partition separately.

A kit could also include media that is already derivatized with antibodies, or has been activated to be derivatized with such antibodies. If a flow plate is used, the kit could contain the flow plate with media ready to apply to the wells. If the partitioned column is used, the kit could include several interlinkable cartridges used for assembly of different kinase columns. The kit also could include substrates for the kinases, appropriate buffers, matrix materials for MS preparation, and probes capable of binding proteins and/or phosphoproteins.

II. Protein Kinases and Affinity Capture

The present invention provides a highly sensitive, accurate determination of one or more protein kinase activities from a single complex biological sample without the need for use of radioactive isotopes or complex purification procedures. The kinase activities are measured by first passing the complex biological sample through an affinity capture apparatus. The apparatus can take a variety of forms, ranging from affinity columns to microtitre wells (see FIG. 2.). Regardless of whether columns or wells are used, the affinity capture apparatus includes a capture reagent that is a molecule capable of specifically recognizing a single kinase or a narrow class of related kinases. In a preferred embodiment, the capture reagent is an antibody, preferably a polyclonal antibody, although embodiments of the invention also contemplate use of monoclonal antibodies to specific kinases or classes of related kinases.

The capture reagent is bound to a solid support, capable of immobilizing the recognized kinase while leaving the remaining components of the biological sample free and available for removal from the capture apparatus. The solid support can be any surface that is insoluble, or capable of being rendered insoluble, in the biological sample containing the kinases being captured. For example, one embodiment utilizes a capture reagent bound to a predominantly planar surface. More preferably, the capture reagent is bound to a microparticle. Still other embodiments utilize solid supports with particular physical properties allowing the support and any associated capture reagent/analyte to be isolated from the biological sample. For example, magnetic microparticles derivatized with specific antibodies can be used. A slurry of derivatized magnetic microparticles can be made from the biological sample. The beads and captured kinase(s) can then be isolated with a magnet.

Each solid support used in the invention can be derivatized with a capture reagent capable of recognizing a single kinase. Alternatively, each solid support can be derivatized with a capture reagent for a number of different kinases (see FIG. 2B). In the latter embodiment, a substrate specific for each kinase is used in the invention. When two or more of the kinase activities to be studied can act on a common substrate, then the former embodiment of the derivatized adsorbent will be used in the invention.

The arrangement of the derivatized solid supports can also take a variety of forms, again depending on the substrate specificities of the kinase activities being studied. For example, when a specific substrate exists for each of the kinase activities being studied, the derivatized solid supports can be mixed together and all kinase capture reactions carried out in a common batch. When two or more of the kinase activities to be studied can act on a common substrate however, each solid support must be derivatized with capture reagent specific for a single kinase or class of related kinases, and the derivatized solid supports must be maintained separate or separable from one another. This latter embodiment does not however preclude capture of all kinases to be studied in a single step. The derivatized solid supports can for example be separated in a single column separated by permeable frits, or more preferably, the derivatized supports can be housed in separate cassettes, each cassette having one or more permeable interfaces for cassettes containing other derivatized supports. By stacking the derivatized supports in a column, with each derivatized support separated by either a permeable frit or cassette interface, the biological sample containing the kinase(s) to be studied can be contacted with all of the derivatized solid supports in a single step, capturing all of the kinases of interest (see FIG. 2).

Another embodiment for maintaining the separation of the derivatized substrates is illustrated in FIG. 2A. By maintaining each derivatized support in a separate compartment, (in FIG. 2 the compartments are in the form of separate wells of a microtiter plate) each kinase, or class of related kinases, can be captured by contacting each derivatized substrate with an aliquot of the biological sample. In a preferred embodiment, the base of each microtiter well comprises a filter membrane sufficient to retain the derivatized solid support within the well, while allowing components of the biological sample that are not captured by the derivatized solid support to pass through. Note that in this preferred embodiment, the run-through components from one derivatized solid support can be used as a source of kinase for a second solid support derivatized with a capture reagent specific for a different kinase.

Suitable biological sources for use in recovering kinase activities to be studied using the present invention include, e.g., body fluids or extracts from biological samples, such as cell lysates. Preferably, the sample is in liquid form and obtained from cell lysates from specific tissues, organs or organisms. The examples below describe kinases captured from mouse brain tissue. Commercially available kinases can be used as needed for controls.

III. Protein Kinase Reactions and Collection of Reactants

As each captured protein kinase exerts its biological effect via an inherent enzyme activity, phosphorylation, it is important to obtain a measurement of that activity in order to appreciate the impact the kinase has in the biological system it is isolated from. To accomplish this, as with all enzyme/substrate systems, it is important to determine the rate and extent of the reaction catalyzed by the enzyme (i.e., the kinase). In the previous section a manner of isolating the protein kinase, e.g., through affinity capture was described in detail. This section will describe how to use the captured kinase to carry out an enzyme-catalyzed reaction

Protein kinase substrates are phosphorylated by contacting them with the requisite protein kinase immobilized on a protein kinase capture reagent. Typically, contacting involves placing the substrate and the immobilized protein kinase in a common buffered solution and incubating the mixture for an appropriate time, allowing phosphorylation to proceed. The solution can be any solution that will support kinase activity toward the added substrate(s). Solutions containing the kinases that are physiologically compatible with kinase reactions are well known. Preferred conditions include physiologically compatible buffers having pH and concentrations of salt known to permit enzymatic reactions. Preferred substrates are specific for particular kinases under evaluation and have different signatures when measured by mass spectrometry. They are generally commercially available. The examples below provide more specifics.

Substrate can be introduced in a variety of ways, for example as a concentrated sample of known concentration. The substrate can be added to kinase bound to a support such as a column matrix formed from the kinase capture reagent, microparticles formed from capture reagent and placed in a multi-well plate, or simply the surfaces of a container within which contact between kinase(s) and substrate(s) is to be performed. Alternatively, the kinase can be removed from the capture reagent prior to contact with substrate(s) and both enzyme and substrate allowed to interact free in solution.

The solution in which kinase and substrate are contacted can be stationary, agitated, or be in controlled flow, such as through a column comprising kinase bound to a support. In the latter embodiment, the substrate is easily harvested at the end of the reaction as the flow-through from the column. In other embodiments, the solution containing the phosphorylated substrate (e.g., kinase product) can be harvested by pipetting off the reaction solution, centrifugation to remove the solid support to which the kinase is immobilized, or other suitable means.

IV. Capturing Phosphoproteins Using Mass Spectrometry-compatible Adsorbents

In one aspect, the invention provides adsorbents on probes where the adsorbents are adapted for use with a mass spectrometer and have the ability to bind phosphorylated proteins. The adsorbent can be in any suitable form as long as it is adapted for use with mass spectrometry and has the capacity to bind the protein kinase product of interest. Such adsorbents include tri- or di-valent chelating metals such as copper, nickel, zinc, or gallium. Typically, the adsorbents are located at addressable positions on the probe, allowing for registered analysis of each addressable position without manipulation of the probe by the user.

Products from protein kinase reactions are generally added to the adsorbent in liquid form, and allowed to bind. The complex is then washed to remove unbound components. EAM material is then added to the adsorbent-bound sample.

The probe can be shaped so that it is adapted for use with various components of the mass spectrometer of the invention, such as inlet systems and detectors. For example, the probe can be adapted for mounting in a horizontally and/or vertically translatable carriage that horizontally and/or vertically moves the probe to a successive position. This allows pathway components bound to different locations of the adsorbent surface to be analyzed without requiring repositioning of the probe by hand. Probes are commercially available and can be purchased (e.g., ProteinChip®, Ciphergen Biosystems, Fremont, Calif.). The probes may be constructed of a variety of materials. Probe materials should not react with sample components, nor should they release molecular species outside those comprising the sample and adsorbents located on their surface as a consequence of exposure to the ionization source of the mass spectrometer. Probe materials include, but are not limited to, insulating materials (e.g., glass such as silicon oxide, plastic, ceramic), semi-conducting materials (e.g., silicon wafers), or electrically conducting materials (e.g., metals, such as nickel, brass, steel, aluminum, gold, or electrically conductive polymers), organic polymers, biopolymers, or any combinations thereof. The probe material can also be solid or porous. Probe materials suitable for use in embodiments of the invention are also described in, e.g., U.S. Pat. No. 5,617,060 (Hutchens and Yip) and WO 98/59360 (Hutchens and Yip).

V. Methods for Detecting Kinase Substrate Components Using Mass Spectrometry

The invention provides methods for detecting protein kinase activities by determining the presence of their phosporylation products. The kinase substrate-containing sample can be applied to an adsorbent on an probe in any manner, e.g., bathing, soaking, dipping, spraying, washing over, or pipetting, etc. Generally, a volume of sample containing from a few attomoles to 100 picomoles in about 1 μl to 500 μl is sufficient for binding to the probe adsorbent. The sample can contact the probe adsorbent comprising capture reagents for a period of time sufficient to allow the pathway components to bind to the capture reagents. Typically, the sample and the adsorbent comprising the capture reagents are contacted for a period of between about 30 seconds and about 12 hours, and preferably, between about 30 seconds and about 15 minutes. Typically, the sample is contacted to the probe adsorbent under ambient temperature and pressure conditions. For some samples, however, modified temperature (typically 4° C. through 37° C.) and pressure conditions can be desirable, which conditions are determinable by those skilled in the art.

After the adsorbent contacts the sample or sample solution, it is preferred that unbound materials on the adsorbent surface are washed out so that only the bound kinase substrate components remain on the adsorbent surface. Washing an adsorbent surface can be accomplished by, e.g., bathing, soaking, dipping, rinsing, spraying, or washing the adsorbent surface with an eluant. A microfluidics process is preferably used when an eluant is introduced to small spots of capture reagents on the probe. Typically, the eluant can be at a temperature of between 0° C. and 100° C., preferably between 4° C. and 37° C.

In some embodiments, washing unbound materials from the probe surface may not be necessary if kinase substrates bound on the probe surface can be resolved by gas phase ion spectrometry without a wash.

Any suitable eluants (e.g., organic or aqueous) can be used to wash the substrate surface. Preferably, an aqueous solution is used. Exemplary aqueous solutions include a HEPES buffer, a Tris buffer, or a phosphate buffered saline, etc. To increase the wash stringency of the buffers, additives can be incorporated into the buffers. These include, but are not limited to, ionic interaction modifier (both ionic strength and pH), water structure modifier, hydrophobic interaction modifier, chaotropic reagents, affinity interaction displacers. Specific examples of these additives can be found in, e.g., PCT publication WO98/59360 (Hutchens and Yip). The selection of a particular eluant or eluant additives is dependent on experimental conditions (e.g., types of capture reagents used or kinase substrate components to be detected), and can be determined by those of skill in the art.

Prior to MS-analysis, an MS-matrix material must be added to the adsorbent-bound sample. The EAM can assist absorption of energy from an ionization source, e.g., from a gas phase ion spectrometer, and can assist desorption of sample components from the probe surface. The MS-matrix can be any suitable material that allows ionization and vaporization of the molecules of interest bound to the adsorbent, and also absorbs some of the ionization source's energy, thereby preventing the ionization source from fragmenting the sample molecules being analyzed. The absorption spectrum of the matrix should overlap the frequency of the laser pulse being used. The matrix should retain stability while at the same time allowing deposition of ionization energy. The matrix also should not react (modify) with the samples to be analyzed and must not sublime away at a rate incompatible with the duration of the analysis. Finally, the matrix should have appropriate chemical properties that allow ionization of the sample material. Common MS-matrix materials include hydroxycinnamic acid (CHCA), sinapinic acid, gentisic acid, trans-3-indoleacrylic acid (IAA), and cinnamic, nicotinic and vanillic acids. See, e.g., U.S. Pat. No. 5,719,060 (Hutchens & Yip) for additional description of MS matrices.

The EAM can be added to the sample containing kinase substrate components in any suitable manner. For example, an EAM is mixed with the sample, and the mixture is placed on the adsorbent surface. In another example, an EAM can be placed on the adsorbent surface prior to contacting the adsorbent surface with the sample. In another example, the sample can be placed on the adsorbent surface prior to contacting the adsorbent surface with an EAM. Then the components bound to the adsorbent surface are desorbed, ionized and detected as described in detail below.

However, in certain embodiments, the biochip is a SEND probe. Optionally, such probes also have bound adsorbents and therefore also function as SEAC probes. In such case, the addition of external EAM is not necessary.

A. Desorption/Ionization and Detection Protein kinase substrate components bound on the adsorbent surface can be desorbed and ionized using mass spectrometry. Any suitable ionizing mass spectrometer, e.g., a gas phase ion spectrometer, can be used as long as it allows the different protein kinase substrates bound by the adsorbent to be resolved. In a typical mass spectrometer, an probe comprising kinase substrate components bound by the adsorbent is introduced into an inlet system of the mass spectrometer. The kinase substrate components are then desorbed by a desorption source such as a laser, fast atom bombardment, high energy plasma, electrospray ionization, thermospray ionization, liquid secondary ion MS, field desorption, etc. The generated desorbed, volatilized species consist of preformed ions or neutrals which are ionized as a direct consequence of the desorption event. Generated ions are collected by an ion optic assembly, and then a mass analyzer disperses and analyzes the passing ions. The ions exiting the mass analyzer are detected by a suitable detector. The detector then translates information of the detected ions into mass-to-charge ratios. Detection of the presence of kinase substrate components will typically involve detection of signal intensity. This, in turn, can reflect the quantity and characteristics of protein kinases that were included or captured in the prior steps. Any of the parts of a mass spectrometer (e.g., a desorption source, a mass analyzer, a detector, etc.) can be combined with other suitable parts described herein or others known in the art in embodiments of the invention.

Preferably, a laser desorption time-of-flight mass spectrometer is used in embodiments of the invention. In laser desorption mass spectrometry, a probe adsorbent comprising kinase substrate components is introduced into an inlet system. The pathway components are desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of pathway components of specific mass to charge ratio.

In another embodiment, an ion mobility spectrometer can be used to detect kinase substrate components. The principle of ion mobility spectrometry is based on different mobility of ions. Specifically, ions of a sample produced by ionization move at different rates, due to their difference in, e.g., mass, charge, or shape, through a tube under the influence of an electric field. The ions (typically in the form of a current) are registered at the detector which can then be used to identify pathway components in a sample. One advantage of ion mobility spectrometry is that it can operate at atmospheric pressure.

B. Analysis of Data

Data generated by desorption and detection of the kinase substrate components can be analyzed using any suitable means. In one embodiment, data is analyzed with the use of a programmable digital computer. The computer program generally contains a readable medium that stores codes. Certain code can be devoted to memory that includes the location of each feature on a probe, the identity of the capture reagents at that feature and the elution conditions used to wash the Adsorbent surface. The computer also contains code that receives as input, data on the strength of the signal at various molecular masses received from a particular addressable location on the probe. This data can indicate the number of kinase substrate components detected, including the strength of the signal generated by each component.

Data analysis can include the steps of determining signal strength (e.g., height of peaks) of kinase substrate components detected and removing “outerliers” (data deviating from a predetermined statistical distribution). The observed peaks can be normalized, a process whereby the height of each peak relative to some reference is calculated. For example, a reference can be background noise generated by instrument and chemicals (e.g., EAM) which is set as zero in the scale. Then the signal strength detected for each kinase substrate component or other biomolecules can be displayed in the form of relative intensities in the scale desired (e.g., 100). Alternatively, a standard may be admitted with the sample so that a peak from the standard can be used as a reference to calculate relative intensities of the signals observed for each kinase substrate component detected.

The computer can transform the resulting data into various formats for displaying. In one format, referred to as “spectrum view or retentate map,” a standard spectral view can be displayed, wherein the view depicts the quantity of kinase substrate component reaching the detector at each particular molecular weight. In another format, referred to as “peak map,” only the peak height and mass information are retained from the spectrum view, yielding a cleaner image and enabling kinase substrate components with nearly identical molecular weights to be more easily seen. In yet another format, referred to as “gel view,” each mass from the peak view can be converted into a grayscale image based on the height of each peak, resulting in an appearance similar to bands on electrophoretic gels. In yet another format, referred to as “3-D overlays,” several spectra can be overlaid to study subtle changes in relative peak heights. In yet another format, referred to as “difference map view,” two or more spectra can be compared, conveniently highlighting pathway components which are up- or down-regulated compared to control. Profiles (spectra) from any two samples may be compared visually. In yet another format, Spotfire Scatter Plot can be used, wherein pathway components that are detected are plotted as a dot in a plot, wherein one axis of the plot represents the apparent molecular weight of the kinase substrate pathway components detected and another axis represents the signal intensity of components detected. For each sample, kinase substrate components that are detected and the amount of components present in the sample can be saved in a computer readable medium. This data can then be compared to a control (e.g., a profile or quantity of pathway components detected in control, e.g., from healthy subjects).

Data generated by mass spectrometry can then be analyzed by a computer software. The software can comprise code that converts signal from the mass spectrometer into computer readable form. The software also can include code that applies an algorithm to the analysis of the signal to determine whether the signal represents a “peak” in the signal corresponding to the kinases being studied. The software also can include code that executes an algorithm that compares signal from a test sample to a typical signal characteristic of “normal” and determines the closeness of fit between the two signals. The software also can include code indicating whether the test sample has a normal profile of the kinases being studied or if it has a defect.

Peak data from one or more spectra can be subject to further analysis by, for example, creating a spreadsheet in which each row represents a particular mass spectrum, each column represents a peak in the spectra defined by mass, and each cell includes the intensity of the peak in that particular spectrum. Various statistical or pattern recognition approaches can applied to the data.

C. Comparing Test Sample Data to Control

Data generated by desorption and detection of kinase substrate components in a test sample can be compared to a control data to determine if the kinase substrate in the test sample is normal. A control data refers to data obtained from comparable samples from a normal cell or person, which or who is known to have no defects in the pathways involving kinases. For each kinase substrate component being analyzed, a control amount of kinases from a normal sample is determined. Preferably, the control amount of each kinase component is determined based upon a significant number of samples taken from normal cells or persons so that it reflects variations of the amount of these components seen in the normal cell or population.

If the test amount of particular kinase components is significantly increased or decreased compared to the control amount of the component, then this is a positive indication that the test sample has a defect in that pathway. For example, if the test amount of a kinase related pathway component is increased or decreased by at least 1.5 fold, 2 fold, 5 fold or 10 fold compared to the control amount, then this is an indication that the test sample has a defect in that pathway. In some circumstances, if defect is severe, certain components of the pathway may be undetectable.

The spectra that are generated in embodiments of the invention can be classified using a pattern recognition process that uses a classification model. In general, the spectra will represent samples from at least two different groups for which a classification algorithm is sought. For example, the groups can be pathological v. non-pathological (e.g., cancer v. non-cancer), drug responder v. drug non-responder, toxic response v. non-toxic response, progressor to disease state v. non-progressor to disease state, phenotypic condition present v. phenotypic condition absent.

In some embodiments, data derived from the spectra (e.g., mass spectra or time-of-flight spectra) that are generated using samples such as “known samples” can then be used to “train” a classification model. A “known sample” is a sample that is pre-classified. The data that are derived from the spectra and are used to form the classification model can be referred to as a “training data set”. Once trained, the classification model can recognize patterns in data derived from spectra generated using unknown samples. The classification model can then be used to classify the unknown samples into classes. This can be useful, for example, in predicting whether or not a particular biological sample is associated with a certain biological condition (e.g., diseased vs. non diseased).

The training data set that is used to form the classification model may comprise raw data or pre-processed data. In some embodiments, raw data can be obtained directly from time-of-flight spectra or mass spectra, and then may be optionally “pre-processed” as described above.

Classification models can be formed using any suitable statistical classification (or “learning”) method that attempts to segregate bodies of data into classes based on objective parameters present in the data. Classification methods may be either supervised or unsupervised. Examples of supervised and unsupervised classification processes are described in Jain, “Statistical Pattern Recognition: A Review”, IEEE Transactions on Pattern Analysis and Machine Intelligence, Vol. 22, No. 1, January 2000.

In supervised classification, training data containing examples of known categories are presented to a learning mechanism, which learns one more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning processes such as CART—classification and regression trees), artificial neural networks such as backpropagation networks, discriminant analyses (e.g., Bayesian classifier or Fischer analysis), logistic classifiers, and support vector classifiers (support vector machines).

A preferred supervised classification method is a recursive partitioning process. Recursive partitioning processes use recursive partitioning trees to classify spectra derived from unknown samples. Further details about recursive partitioning processes are provided in U.S. 2002 0138208 A1 (Paulse et al., “Method for analyzing mass spectra,” Sep. 26, 2002.

In other embodiments, the classification models that are created can be formed using unsupervised learning methods. Unsupervised classification attempts to learn classifications based on similarities in the training data set, without pre classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

Learning algorithms asserted for use in classifying biological information are described in, for example, WO 01/31580 (Barnhill et al., “Methods and devices for identifying patterns in biological systems and methods of use thereof,” May 3, 2001); U.S. 2002 0193950 A1 (Gavin et al., “Method or analyzing mass spectra,” Dec. 19, 2002); U.S. 2003 0004402 A1 (Hitt et al., “Process for discriminating between biological states based on hidden patterns from biological data,” Jan. 2, 2003); and U.S. 2003 0055615 A1 (Zhang and Zhang, “Systems and methods for processing biological expression data” Mar. 20, 2003).

The classification models can be formed on and used on any suitable digital computer. Suitable digital computers include micro, mini, or large computers using any standard or specialized operating system such as a Unix, Windows™ or Linux™ based operating system. The digital computer that is used may be physically separate from the mass spectrometer that is used to create the spectra of interest, or it may be coupled to the mass spectrometer.

The training data set and the classification models according to embodiments of the invention can be embodied by computer code that is executed or used by a digital computer. The computer code can be stored on any suitable computer readable media including optical or magnetic disks, sticks, tapes, etc., and can be written in any suitable computer programming language including C, C++, visual basic, etc.

Thus, for example, a collection of samples corresponding to one form of a biological state (e.g., pathologic) can be compared with a collection of samples corresponding to another form of the biological state (e.g., non-pathologic). A learning algorithm, as described above, can detect patterns of peaks, corresponding to, for example, the presence or absence of the activity of a plurality of kinases, that can classify an unknown sample as belonging to one form of the biological state. The pattern of markers characteristic of the pathological state may, in turn, indicate a particular pathway that is functioning in an abnormal manner. Such faulty kinases may be drug targets for drug discovery, or their replacement with a normal kinase may be a drug or the product they normally produce may be a drug.

VI. Kits

Another aspect of the invention provides kits comprising a protein kinase substrate(s) adapted for use with a mass spectrometer described herein and an instruction material for using the kit to detect kinase activities. Alternative kits comprise probes optionally including different adsorbent surface chemistries, antibodies specific for particular kinases derivatized to a solid support, compatible MS matrices and/or “control” protein kinase preparations. The kits of the invention have many applications. For example, the kits can be used to determine if a test sample has normal or defective kinase activity that is or is not related to an important pathway. In another example, the kits can be used to identify compounds that modulate the expression of one or more kinases associated in a cellular pathways in vitro or in vivo.

In one embodiment, a kit comprises: (a) an adsorbent adapted for use with a mass spectrometer, the adsorbent comprises immobilized capture reagents on an probe, wherein the capture reagents specifically bind to the different substrates of various kinases being studied; and (b) instruction material for detecting the different kinase substrates by contacting a sample containing different kinase substrates with the adsorbent and detecting the kinase substrates retained by the capture reagents. Instruction material can be in the form of a label on the package or separate insert material.

In some embodiments, the kit may comprise an eluant (as an alternative or in combination with an instruction material) for washing the adsorbent, which eluant allows retention of kinase substrate components when washed with eluant. Alternatively or additionally, the kit may further comprise an instruction material for washing the adsorbent with the eluant after contacting the adsorbent with a sample. Such kits can be prepared from the materials described above, and the previous discussion of these materials (e.g., probe adsorbents, capture reagents, washing solutions, etc.) is fully applicable to this section and will not be repeated.

Optionally, the kit may further comprise standard or control information so that the test sample can be compared with the control information standard to determine if the kinase activities being studied in a test sample are normal and/or the assay for these activities is functioning properly. For example, standards include samples of known kinase activities free from contaminating kinase activities and, where necessary, contaminating proteins that could be potential substrates for sample kinases.

The present invention provides novel materials and methods for detecting kinase substrates using mass spectroscopy. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

All publications and patent documents cited in this application are incorporated by reference in their entirety for all purposes to the same extent as if each individual publication or patent document were so individually denoted. By their citation of various references in this document, Applicants do not admit any particular reference is “prior art” to their invention.

### EXAMPLES

In this example, we describe a method of monitoring kinase activity by surface enhanced laser desorption/ionization (SELDI) mass spectrometry. It is reasonably sensitive, it eliminates the necessity for the use of radioactivity, and it can be easily multiplexed for the analysis of several different kinases in a single reaction mixture. We have compared the SELDI-based kinase assay to the conventional radioactivity assay by determining kinase activities in mouse brain lysates. In addition, we have used the SELDI kinase assay to determine EC₅₀ of kinase activities and IC₅₀ of kinase inhibitors in a high-throughput fashion. Furthermore, we have mapped phosphorylation sites by using a combination of SELDI-based phosphopeptide detection and MS/MS analysis.

Materials:

All kinases (AKT1, JNK1α1, c-Src and GSK3β kinase) and their corresponding substrates were supplied as a courtesy of Upstate Discovery. PKC α and its substrate and inhibitor were purchased from Upstate Biotechnology. All ProteinChip® arrays, alphacyano-4-hydroxycinamic acid (CHCA), sinapinic acid (SPA) were obtained from Ciphergen Biosystems, Inc. Mouse brain lysates were a gift of Dr. L Mucke at Gladstone Institute of Neurological Diseases.

Kinase Assay:

In a final reaction volume of 25 μl, a kinase (5-50 mUnits, Upstate Discovery) was incubated with the corresponding substrate (3-250 mM, Upstate Discovery) in a buffer containing 1 mM ATP for 30 min at 30oC. 10 μl of the reaction mix was analyzed on gallium-immobilized metal affinity capture (IMAC-Ga) ProteinChip® arrays as described below. Multiplexed kinase assay was performed by mixing multiple kinases and their substrates in a single tube. For mouse brain Fyn kinase assay, Fyn was immunoprecipitated with anti-Fyn antibody from mouse brain lysates (The Gladstone Institutes) and assayed with the c-Src substrate as described above. For PKCα activity titration assays, each 25 μl reaction contained 50 mM substrate; and for PKCα inhibition curve, 0.12 ng/μ l of PKCα was used. These reactions were carried out for 6 min at RT.

Capture of Phosphopeptide using IMAC-Ga ProteinChip® Arrays:

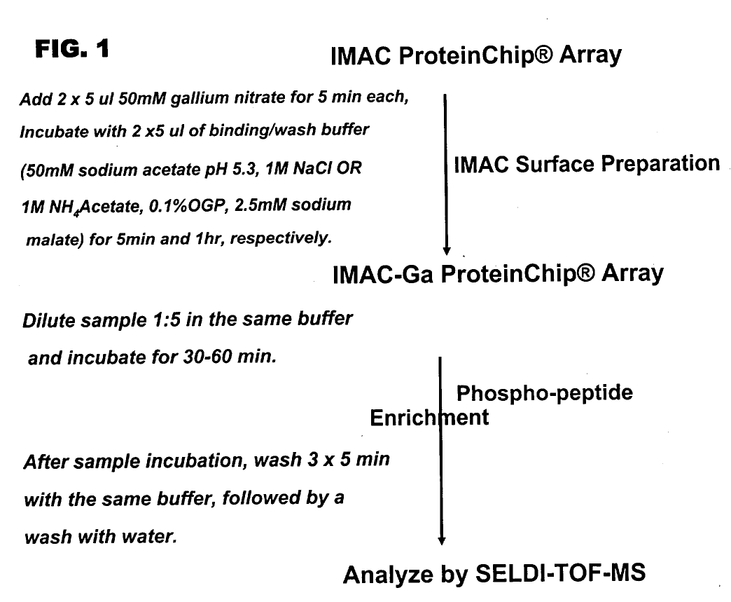

The phosphorylated kinase substrates were captured and enriched for detection using IMAC-Ga ProteinChip® arrays as outlined in FIG. 1.

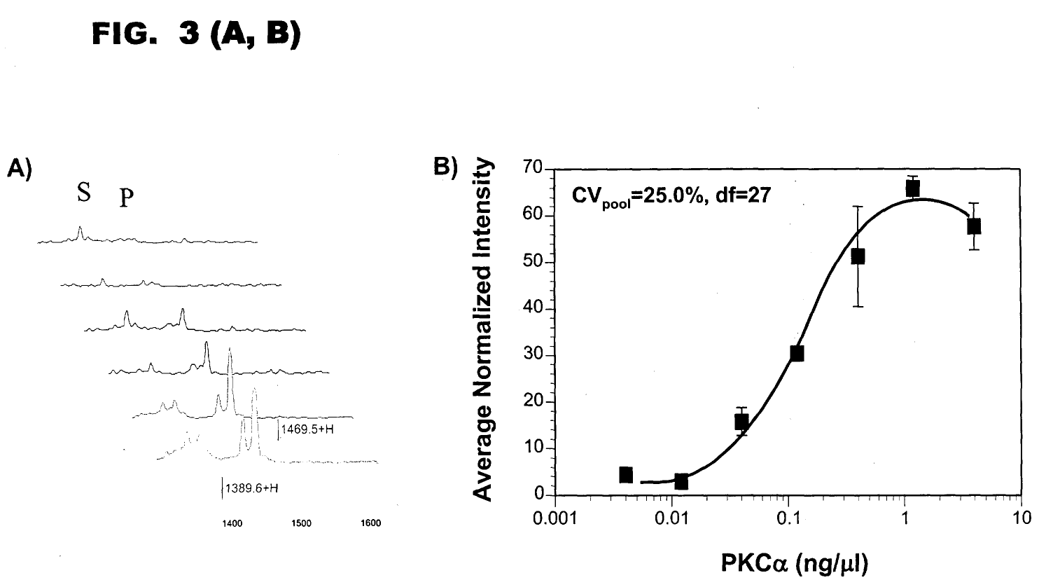

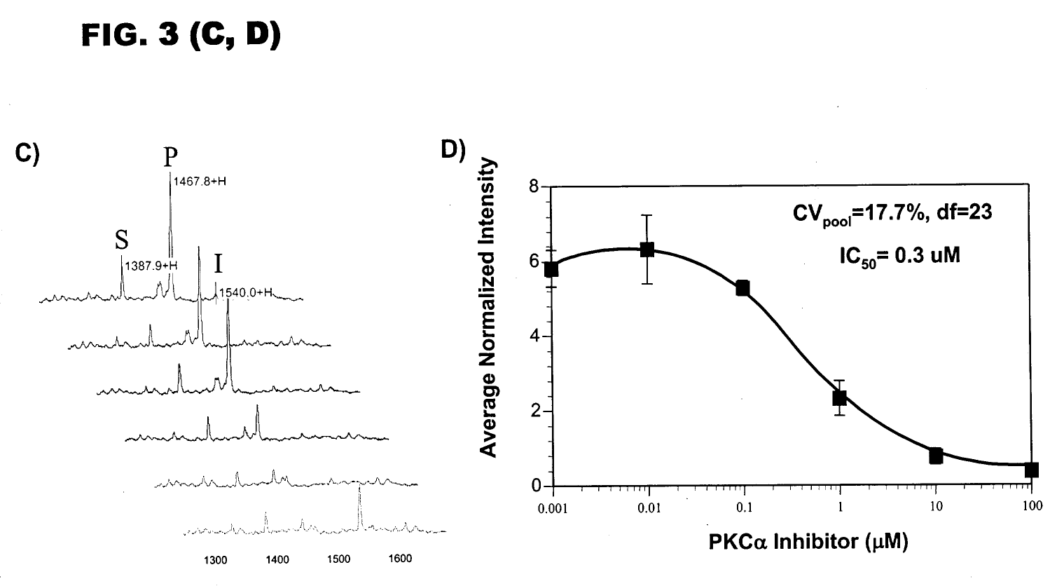

FIG. 2 provides an overview of the invention carried out in a multi-well plate containing different kinases exposed to different kinase substrates. FIGS. 3A-C demonstrates the facility of SELDI MS to detect the phosphorylated substrate of PKCα including the activity of the enzyme in the presence or absence of a PKCα inhibitor.

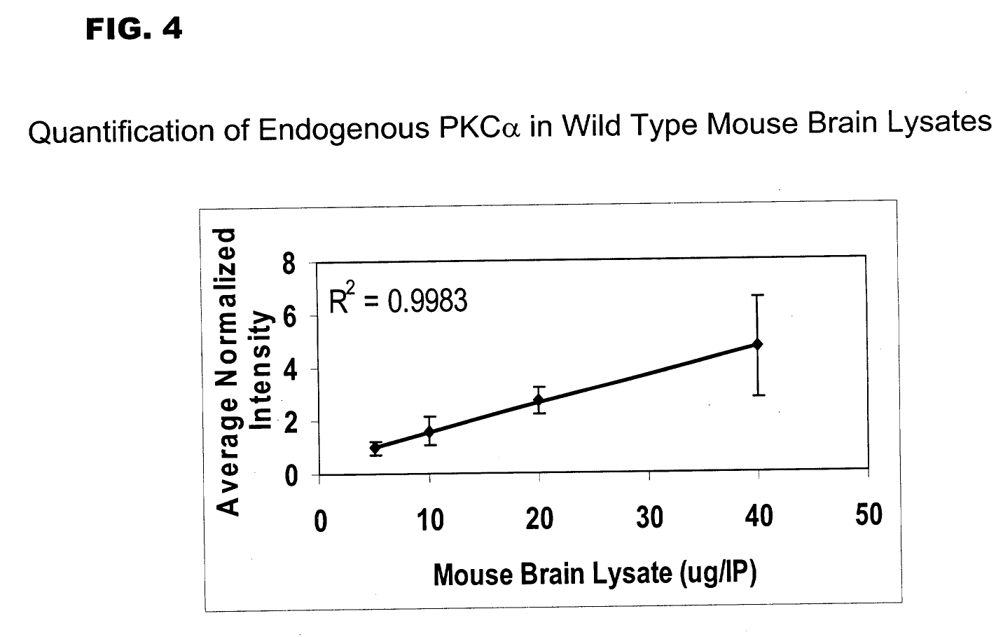

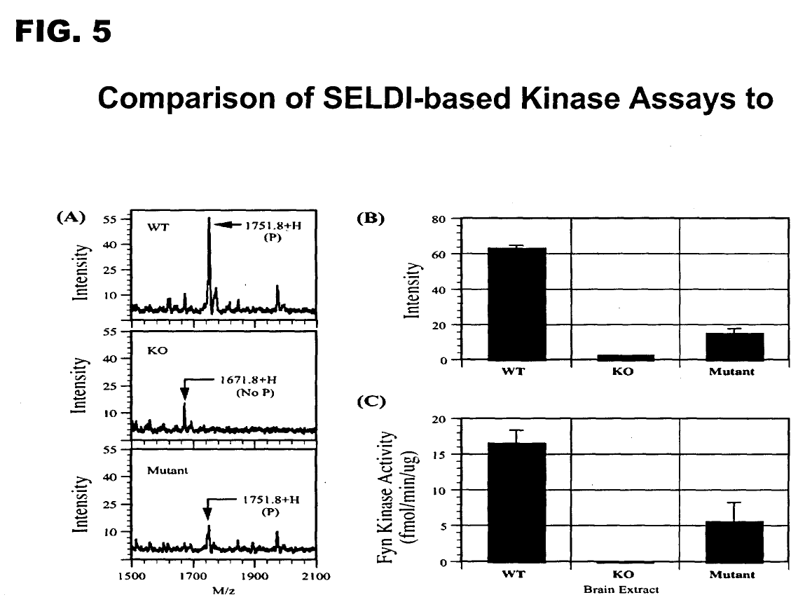

The utility of the invention to work in a real world environment is exemplified by FIG. 4 where a brain lysate of mouse was analyzed for the presence of PKCα. FIG. 5 provides useful comparison of the relative sensitivity of the SELDI-MS approach to conventional radioactivity assays for detection of kinase substrates. In this case the kinase was Fyn kinase using the cSrc substrate.

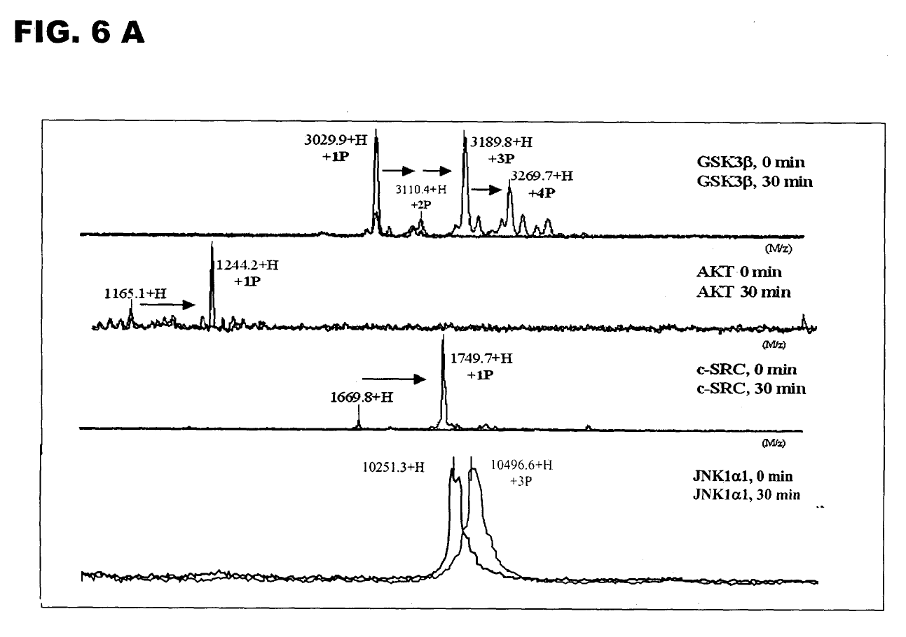

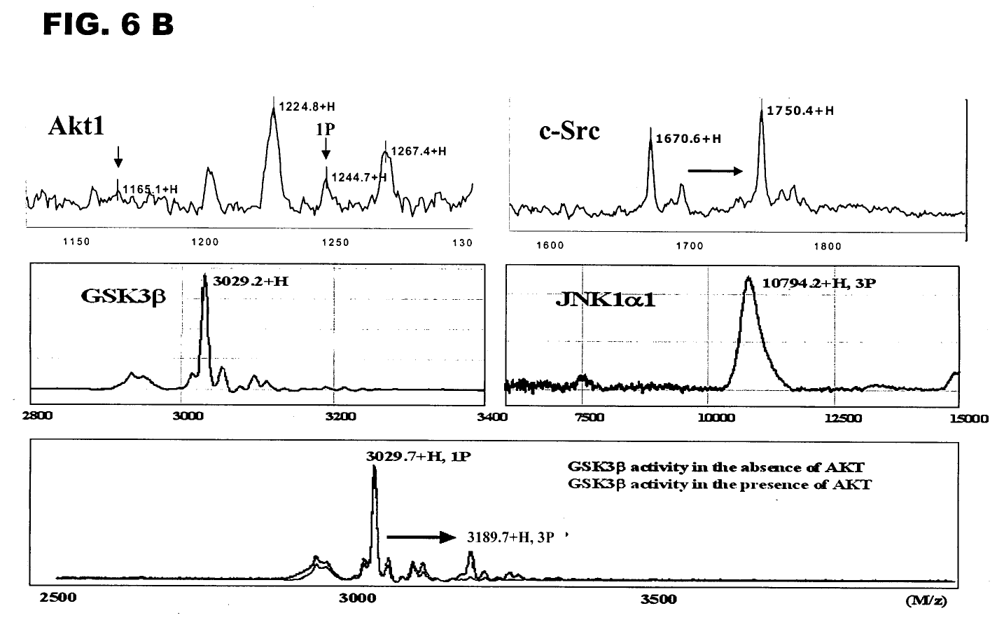

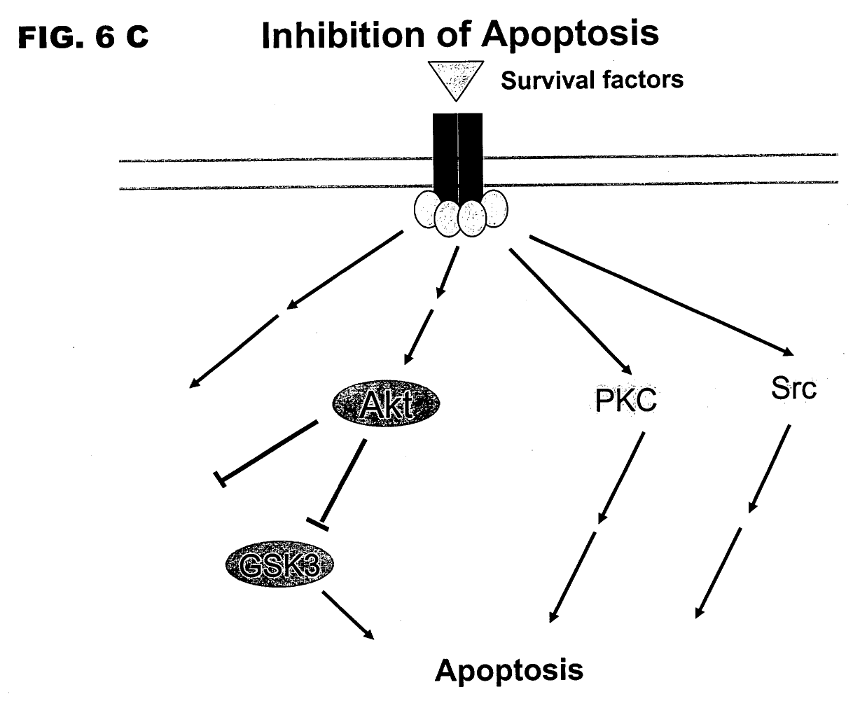

FIGS. 6A-C describes a multiplex assay where 4 different kinases are evaluated using the MS system described here. In FIG. 6A, the four different kinases involved in apoptosis are being studied using 4 separate substrates. The kinases and the substrates have been combined and analyzed together. In FIG. 6B, the phosphorylation of the individual substrates is described. In the lowest box a comparison of the effect of Akt1 on GSK3β is described. Akt1 can inactivate GSK3β and this is clearly evident in the data presented. The cartoon of FIG. 6C illustrates the potency of batch analysis to evaluate cellular pathways involving kinases and their cascades. Briefly, a single reaction mixture containing the kinases GSK3β, AKT, c-SRC and JNK1α1 and their substrates was prepared. An aliquot of this reaction mix was taken prior to addition of ATP and analyzed using the SELDI technique. ATP was then added to the reaction mix and the reaction allowed to proceed for 30 minutes. The mass determinations from both time points are depicted as superimposed in the figure.

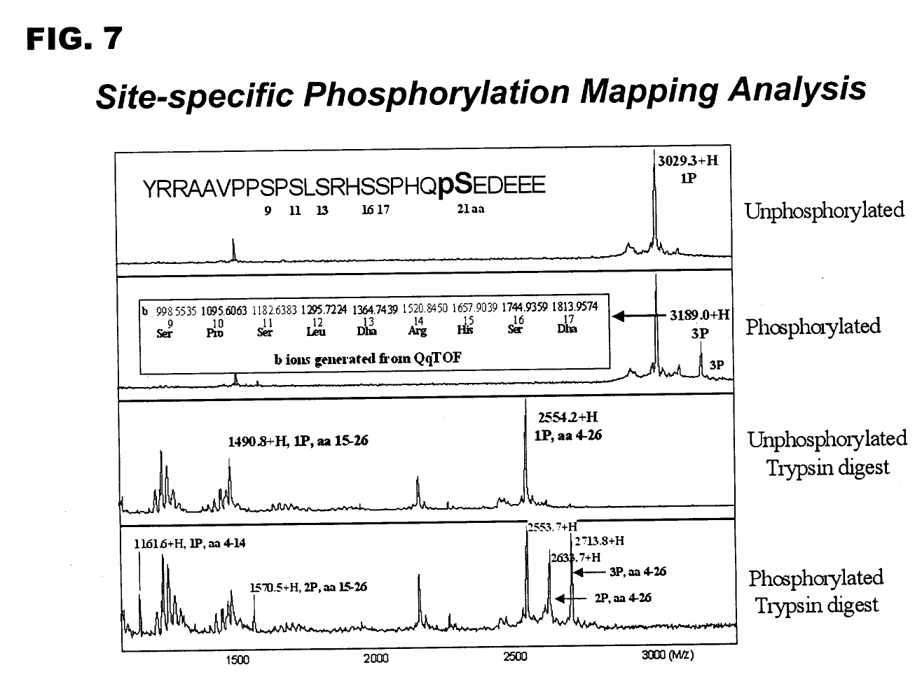

Finally, FIG. 7, illustrates that one can use mass spectroscopy to track phosphorylation to specific sites in a kinase substrate. Phosphorylated and unphosphorylated forms of GSK3β substrate were treated with trypsin for one hour. Peptides were analyzed both on PBSii and QqTOF from Micromass equipped with Ciphergen Proteinchip® array interface.

What is claimed is:

1. A method for detecting protein kinases in a sample, the method comprising:
a. contacting the sample with a plurality of affinity molecules, each affinity molecule specific for a different protein kinase, whereby the protein kinases of the sample are bound to the affinity molecules;
b. immobilizing the bound protein kinases on a solid support;
c. washing the immobilized protein kinases, thereby separating unbound material from the protein kinases;
d. contacting each of the washed, immobilized protein kinases with a substrate recognized by the kinase, thereby converting the substrate to a phosphorylated product, wherein the phosphorylated product of each kinase has a unique mass signature;
e. capturing the phosphorylated products on an probe comprising an adsorbent surface that binds the phosphorylated products; and
f. detecting the phosphorylated products bound to the probe by mass spectrometry, whereby detecting the mass signature of the phosphorylated product of a protein kinase indicates the presence of the protein kinase in the sample.

2. The method of claim 1 wherein the affinity molecules are immobilized on the solid supports before contacting the sample with the affinity molecules.

3. The method of claim 1 wherein the affinity molecules are immobilized on the solid support after binding the protein kinases.

4. The method of claim 2 or 3 wherein each solid support bears a plurality of different affinity molecules that each recognize a different protein kinase.

5. The method of claim 2 or 3 wherein each solid support bears affinity molecules that recognize only one protein kinase.

6. The method of claim 5 wherein step (d) comprises contacting the protein kinases in a common flow path, wherein the solid supports bearing affinity molecules recognizing a particular protein kinase are segregated in the flow path from the solid supports recognizing different protein kinases.

7. The method of claim 5 wherein step (d) comprises contacting the protein kinases in a common flow path, wherein the solid supports bearing affinity molecules recognizing a particular protein kinase are not segregated in the flow path from the solid supports recognizing different protein kinases.

8. The method of claim 5 wherein step (d) comprises contacting solid supports bearing affinity molecules recognizing a particular protein kinase in different flow paths.

9. The method of claim 8 wherein the solid supports are segregated in different wells of a multi-well flow plate.

10. The method of claim 1 wherein the affinity molecules are antibodies.

11. The method of claim 2 wherein the affinity molecules are antibodies.

12. The method of claim 3 wherein the affinity molecules are antibodies and the solid supports comprise protein A or Protein G bound thereto.

13. The method of claim 1 wherein the insoluble support of the associating step is a microparticle, or a plastic surface.

14. The method of claim 1 wherein the adsorbent surface comprises a metal atom chelate.

15. The method of claim 14 wherein the metal atom chelate comprises copper, nickel, zinc, or gallium.

16. The method of claim 1 wherein the probe comprises a plurality of addressable locations, each addressable location comprising an adsorbent surface.

17. The method of claim 1 wherein the affinity molecules are specific for a plurality of different protein kinases wherein the protein kinases are components of a signal transduction pathway.

18. The method of claim 17 wherein the signal transduction pathway is an apoptosis pathway.

19. A kit for detecting protein kinases in a sample comprising
a) a plurality of solid supports, each solid support having means to bind, or having bound thereto, an affinity molecule specifically recognizing a different protein kinase;
b) a plurality of substrates, each substrate recognized by a different protein kinase recognized by one of the affinity molecules, wherein phosphorylation of a substrate by a protein kinase produces a phosphorylated product with a unique mass signature; and
c) an probe having a adsorbent surface that binds the phosphorylated products.

20. The kit of claim 19 further comprising instruction material comprising details for using the kit.

21. The kit of claim 19 wherein the affinity molecules are antibodies.

22. The kit of claim 19 wherein the solid supports have means to bind the affinity molecules.

23. The kit of claim 22 wherein the affinity molecules are antibodies and the means comprise protein A or protein G.

24. The kit of claim 19 wherein the solid supports have affinity molecules bound thereto.

25. The kit of claim 24 wherein each solid support bears a plurality of different affinity molecules that each recognize a different protein kinase.

26. The kit of claim 24 wherein each solid support bears affinity molecules that recognize only one protein kinase.

27. The kit of claim 24 wherein the solid supports are in stackable cartridges.

28. The kit of claim 19 further comprising a flow column capable of accepting the solid supports.

29. The kit of claim 24 comprising a multi-well flow plate wherein each well is capable of accepting the solid supports.

30. The kit of claim 19 wherein the adsorbent surface comprises a metal atom chelate.

31. The kit of claim 27 wherein the metal atom chelate comprises copper, nickel, zinc, or gallium.
